(19) United States
(12) Patent Application Publication
(10) Pub. No.: US 2003/0073644 A1
(43) Pub. Date: Apr. 17, 2003

(54) Combinations comprising a beta-agonist and a further antidiabetic agent

(75) Inventor: Jonathan Robert Sanders Arch, Welwyn Garden City (GB)

Correspondence Address: GLAXOSMITHKLINE, Corporate Intellectual Property - UW 2220, P.O. Box 1539, King of Prussia, PA, 19406-0939, US

(73) Assignee: SmithKline Beecham p.l.c.

(21) Appl. No.: 10/243,164

(22) Filed: Sep. 13, 2002

Related U.S. Application Data
(63) Continuation of application No. 09/831,651, filed on Jul. 11, 2001, now abandoned.
Application No. 09/831,651 is a 371 of international application No. PCT/GB99/03755, filed on Nov. 11, 1999.

(30) Foreign Application Priority Data
Nov. 11, 1998 (GB) 9824789.3
Nov. 11, 1998 (GB) 9824791.9
Nov. 11, 1998 (GB) 9824790.1

(51) Int. Cl. A61K 31/70; A61K 31/4439; A61K 31/426

(52) U.S. Cl. 514/25; 514/342; 514/369

(57) ABSTRACT

A method for the treatment of diabetes mellitus and conditions associated with diabetes mellitus in a mammal such as a human, which method comprises administering an effective, non-toxic and pharmaceutically acceptable amount of a beta agonist and another antidiabetic agent, to a mammal in need thereof.

This invention relates to a method of treatment, in particular to a method for the treatment of diabetes mellitus, especially non-insulin dependent diabetes (NIDDM) or Type 2 diabetes and conditions associated with diabetes mellitus and to compositions for use in such method.

Alpha glucosidase inhibitor antihyperglycaemic agents (or alpha glucosidase inhibitors) and biguanide antihyperglycaemic agents (or biguanides) are commonly used in the treatment of Type 2 diabetes. Acarbose, voglibose, emiglitate and miglitol are examples of alpha glucosidase inhibitors. 1,1-Dimethylbiguanidine (or metformin) is a particular example of a biguanide.

Insulin secretagogues are compounds that promote increased secretion of insulin by the pancreatic beta cells. The sulphonylureas are well known examples of insulin secretagogues. The sulphonylureas act as hypoglycaemic agents and are used in the treatment of Type 2 diabetes. Examples of sulphonylureas include glibenclamide (or glyburide), glipizide, gliclazide, glimepiride, tolazamide and tolbutamide.

European Patent Application, Publication Number 0,306,228 relates to certain thiazolidinedione derivatives disclosed as having antihyperglycaemic and hypolipidaemic activity. One particular thiazolidinedione disclosed in EP 0306228 is 5-[4-[2-(N-methyl-N-(2-pyridyl)amino)ethoxy]benzyl]thiazolidine-2,4-dione (hereinafter ‘Compound (I)’). WO94/05659 discloses certain salts of Compound (1) including the maleate salt at example 1 thereof.

Compound (I) is an example of a class of anti-hyperglycaemic agents known as ‘insulin sensitisers’. In particular Compound (I) is a thiazolidinedione insulin sensitiser. Compound (I) is also a peroxisome proliferator-activated receptor (PPARγ) agonist insulin sensitiser.

European Patent Applications, Publication Numbers: 0008203, 0139421, 0032128, 0428312, 0489663, 0155845, 0257781, 0208420, 0177353, 0319189, 0332331, 0332332, 0528734, 0508740; International Patent Application, Publication Numbers 92/18501, 93/02079, 93/22445 and U.S. Pat. Nos. 5,104,888 and 5,478,852, also disclose certain thiazolidinedione insulin sensitisers.

Another series of compounds generally recognised as having insulin sensitiser activity are those typified by the compounds disclosed in International Patent Applications, Publication Numbers WO93/21166 and WO94/01420. These compounds are herein referred to as ‘acyclic insulin sensitisers’. Other examples of acyclic insulin sensitisers are those disclosed in U.S. Pat. No. 5,232,945 and International Patent Applications, Publication Numbers WO92/03425 and WO91/19702.

Examples of other insulin sensitisers are those disclosed in European Patent Application, Publication Number 0533933, Japanese Patent Application Publication Number 05271204 and U.S. Pat. No. 5,264,451.

International Patent Application, Publication Number WO97/25311 and European patent application publicaiton number EP0882707A1 relate to certain ethanolamine derivatives of formula (I):

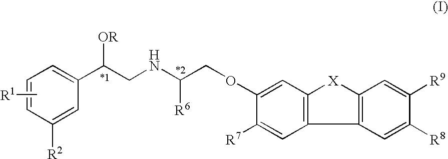

or a salt thereof, in which R represents hydrogen atom or methyl, R¹ stands for hydrogen atom, halogen atom, hydroxy, benzyloxy, amino or hydroxymethyl, R² stands for hydrogen atom, hydroxymethyl, NHR³, SO₂NR⁴R^{4,} or nitro, wherein R³ is hydrogen atom, methyl, SO₂R⁵, formyl or CONHR^{6,}, with R⁵ being a lower alkyl, benzyl or NR⁴R^{4,} and R^{6,} being hydrogen atom or lower alkyl, and R⁴ and R^{4,} may be identical with or different from each other and stand each for hydrogen atom, lower alkyl or benzyl, R⁶ represents hydrogen atom or lower alkyl, X stands for a secondary nitrogen atom, oxygen atom, sulfur atom or methylene and, in case X is secondary nitrogen atom, oxygen atom or sulfur atom, R⁹ stands for hydrogen atom and either one of R⁷ and R⁸ is hydrogen atom and the other one is hydrogen atom, amino, acetylamino or hydroxy, or, in case X is methylene, both R⁷ and R⁸ are hydrogen atom and R⁹ stands for hydrogen atom, amino, acetylamino or hydroxy, *1 indicates an asymmetric carbon atom and *2 indicates that the carbon atom is asymmetric provided that R⁶ is lower alkyl.

The compounds of WO97/25311 and EP0882707A1 are stated to have beta 3 adrenoreceptor agonist activity and are disclosed as being useful for the treatment and prevention of diabetes, hyperlipidaemia and obesity.

The above mentioned publications are incorporated herein by reference.

It is now indicated that the beta 3 adrenoceptor agonist compounds of WO97/25311 and EP0882707A1 in combination with other antidabetic agents provide a particularly beneficial effect on glycaemic control and that such combination is therefore suggested to be particularly useful for the treatment of diabetes mellitus, especially Type 2 diabetes and conditions associated with diabetes mellitus. Such combinations will provide improved blood glucose regulation without introducing unacceptable side-effects. In particular, combination of the beta 3-adrenoceptor agonist with other antidiabetic agents (especially sulphonylureas, insulin sensitisers or insulin) is expected to ameliorate the body weight increasing effects of the other antidiabetic agents. Moreover, it is considered that the beneficial thermogenic effects of the beta-3 agonist will be enhanced in the combination. Thus for example the weight reducing effects of the beta-3 agonist are expected to be enhanced.

Accordingly, the invention provides a method for the treatment of diabetes mellitus, especially Type 2 diabetes and conditions associated with diabetes mellitus in a mammal such as a human, which method comprises administering an effective, non-toxic and pharmaceutically acceptable amount of a beta agonist and another antidiabetic agent, to a mammal in need thereof.

In another aspect the invention provides a beta agonist and another antidiabetic agent, for use in a method for the treatment of diabetes mellitus, especially Type 2 diabetes and conditions associated with diabetes mellitus.

The method comprises either co-administration of a beta agonist and another antidiabetic agent or the sequential administration thereof.

Co-administration includes administration of a formulation which includes both a beta agonist and the other antidiabetic agent or the essentially simultaneous administration of separate formulations of each agent.

In another aspect the invention provides the use of a beta agonist and another antidiabetic agent for use in the manufacture of a composition for the treatment of obesity, diabetes mellitus, especially Type 2 diabetes and conditions associated with diabetes mellitus.

Suitably, the other antidiabetic agent is selected from an alpha glucosidase inhibitor, a biguanide, an insulin secretagogue or an insulin sensitiser.

A further suitable antidiabetic agent is insulin.

A suitable alpha glucosidase inhibitor is acarbose.

Other suitable alpha glucosidase inhibitors are emiglitate and miglitol. A further suitable alpha glucosidase inhibitor is voglibose.

Suitable biguanides include metformin, buformin or phenformin, especially metformin.

Suitable insulin secretagogues include sulphonylureas.

Suitable sulphonylureas include glibenclamide, glipizide, gliclazide, glimepiride, tolazamide and tolbutamide. Further sulphonylureas include acetohexamide, carbutamide, chlorpropamide, glibomuride, gliquidone, glisentide, glisolamide, glisoxepide, glyclopyamide and glycylamide. Also included is the sulphonylurea glipentide.

A further suitable insulin secretagogue is repaglinide. An additional insulin secretagogue is nateglinide.

Insulin sensitisers include PPARγ agonist insulin sensitisers. Insulin sensitisers also include thiazolidinedione insulin sensitisers.

A preferred insulin sensitiser is Compound (I) or a derivative therof.

Other suitable thiazolidinedione insulin sensitisers include (+)-5-[[4-[(3,4-dihydro-6-hydroxy-2,5,7,8-tetramethyl-2H-1-benzopyran-2-yl)methoxy]phenyl]methyl]-2,4-thiazolidinedione (or troglitazone), 5-[4-[(1-methylcyclohexyl)methoxy]benzyl]thiazolidine-2,4-dione (or ciglitazone), 5-[4-[2-(5-ethylpyridin-2-yl)ethoxy]benzyl]thiazolidine-2,4-dione (or pioglitazone) or 5-[(2-benzyl-2,3-dihydrobenzopyran)-5-ylmethyl)thiazolidine-2,4-dione (or englitazone).

A particular thiazolidinedione insulin sensitiser is 5-[4-[2-(5-ethylpyridin-2-yl)ethoxy]benzyl]thiazolidine-2,4-dione (or pioglitazone).

A particular thiazolidinedione insulin sensitiser is (+)-5-[[4-[(3,4-dihydro-6-hydroxy-2,5,7,8-tetramethyl-2H-1-benzopyran-2-yl)methoxy]phenyl]methyl]-2,4-thiazolidinedione (or troglitazone).

Particular, beta agonists are selective for the human beta 3 receptor. Particular, beta agonists have minimal effects upon human beta 1 and/or beta 2 receptors, thereby producing minimal beta 1 and/or beta 2 mediated side-effects. Beta 2 mediated side-effects include tremor, hypokalemia and vasodilatation, which can result in tachycardia. Beta 1 mediated side-effects include tachycardia.

A suitable beta agonst is a compound of formula (I) of WO19725311.

Particular beta agonists include the specific examples disclosed in WO/9725311 and EP0882707A1, which documents are incorporated herein by reference.

A particular compound of WO/9725311 or EP0882707A1 is selected from the list consisting of: (R)-N-[5-[2-[2-(9H-carbazol-2-yloxy)ethylamino]-1-hydroxyethyl]-2-hydroxyphenyl]methanesulfonamide,

(S)-N-[5-[2-[2-(9H-carbazol-2-yloxy)ethylamino]-1-hydroxyethyl]-2-hydroxyphenyl]methanesulfonamide,

N-[5-[2-[2-(9H-carbazol-2-yloxy)ethylamino]-1-hydroxyethyl]-2-hydroxyphenyl]methanesulfonamide,

N-[5-[2-[2-(3-hydroxy-9H-carbazol-2-yloxy)ethylamino]-1-hydroxyethyl]-2-hydroxyphenyl]methanesulfonamide,

N-[5-[2-[2-(3-amino-9H-carbazol-2-yloxy)ethylamino]-1-hydroxyethyl]-2-hydroxyphenyl]methanesulfonamide,

N-[5-[2-[2-(6-amino-9H-carbazol-2-yloxy)ethylamino]-1-hydroxyethyl]-2-hydroxyphenyl]methanesulfonamide,

N-[5-[2-[2-(6-hydroxy-9H-carbazol-2-yloxy)ethylaminol-1-hydroxyethyl]-2-hydroxyphenyl]methanesulfonamide,

(R)-N-[3-[2-[2-(9H-carbazol-2-yloxy)ethylamino]-1-hydroxyethyl]phenyl]methanesulfonamide,

(S)-N-[3-[2-[2-(9H-carbazol-2-yloxy)ethylamino]-1-hydroxyethyl]phenyl]methanesulfonamide,

N-[3-[2-[2-(9H-carbazol-2-yloxy)ethylamino]-1-hydroxyethyl]phenyl]methanesulfonamide,

N-methyl-3-[2-[2-(9H-carbazol-2-yloxy)ethylamino]-1-hydroxyethyl]benzenesulfonamide,

N-methyl-[5-[2-[2-(9H-carbazol-2-yloxy)ethylamino]-1-hydroxyethyl]-2-hydroxy]benzenesulfonamide,

(R)-N-[5-[2-[2-(dibenzofuran-3-yloxy)ethylamino]-1-hydroxyethyl]-2-hydroxyphenyl]methanesulfonamide,

(S)-N-[5-[2-[2-(dibenzofuran-3-yloxy)ethylamino]-1-hydroxyethyl]-2-hydroxyphenyl]methanesulfonamide,

N-[5-[2-[2-(dibenzofuran-3-yloxy)ethylamino]-1-hydroxyethyl]-2-hydroxyphenyl]methanesulfonamide,

N-[5-[2-[2-(9H-carbazol-2-yloxy)ethylamino]-1-hydroxyethyl]-2-(benzyloxy)phenyl]methanesulfonamide,

N-[5-[2-[2-(9H-carbazol-2-yloxy)ethylamino]-1-hydroxyethyl]-2-chlorophenyl]methanesulfonamide,

N-[5-[2-[2-(9H-carbazol-2-yloxy)ethylamino]-1-hydroxyethyl]-2-fluorophenyl]methanesulfonamide,

N-[3-[2-[2-(dibenzofuran-3-yloxy)ethylamino]-1-hydroxyethyl]phenyl]methanesulfonamide,

N-[5-[2-[2-(7-acetylaminofluoren-2-yloxy)ethylamino]-1-hydroxyethyl]-2-hydroxyphenyl]methanesulfonamide,

N-[5-[2-[2-(7-aminofluoren-2-yloxy)ethylamino]-1-hydroxyethyl]-2-hydroxyphenyl]methanesulfonamide,

N-[3-[2-[2-(7-acetylaminofluoren-2-yloxy)ethylamino]-1-hydroxyethyl]phenyl]methanesulfonamide,

N-[3-[2-[2-(7-aminofluoren-2-yloxy)ethylamino]-1-hydroxyethyl]phenyl]methanesulfonamide,

N-[5-[2-[2-(9H-carbazol-2-yloxy)ethylamino]-1-hydroxyethyl]-2-hydroxyphenyl]formamide,

N-[3-[2-[2-(9H-carbazol-2-yloxy)ethylamino]-1-hydroxyethyl]phenyl]formamide,

N-[3-[2-[[1-(9H-carbazol-2-yloxy)propan-2R-yl]amino]-1-hydroxyethyl]phenyl]methanesulfonamide,

2-[N-[2-(9H-carbazol-2-yloxy)ethyl]amino]-1-(4-hydroxy-3-nitrophenyl)ethanol,

2-[N-[2-(9H-carbazol-2-yloxy)ethyl]amino]-1-(3-amino-4-hydroxyphenyl)ethanol,

N-[5-[2-[2-(9H-carbazol-2-yloxy)ethylamino]-1-hydroxyethyl]-2-(benzyloxy)phenyl]urea,

N-[5-[2-[2-(9H-carbazol-2-yloxy)ethylamino]-1-hydroxyethyl]-2-hydroxyphenyl]urea,

N-[5-[2-[2-(9H-carbazol-2-yloxy)ethylamino]-1-hydroxyethyl]-2-(benzyloxy)phenyl]formamide,

N′-[5-[2-[2-(9H-carbazol-2-yloxy)ethylamino]-1-hydroxyethyl]-2-(benzyloxy)phenyl]-N,N-dimethylsulfamide,

N′-[5-[2-[2-(9H-carbazol-2-yloxy)ethylamino]-1-hydroxyethyl]-2-hydroxyphenyl]-N,N-dimethylsulfamide,

2-[N-[2-(9H-carbazol-2-yloxy)ethyl]amino]-1-[3-(methylamino)-4-(benzyloxy)phenyl]ethanol,

2-[N-[2-(9H-carbazol-2-yloxy)ethyl]amino]-1-[3-(methylamino)-4-hydroxyphenyl]ethanol,

N-[5-[2-[2-(9H-carbazol-2-yloxy)ethylamino]-1-hydroxyethyl]-2-hydroxyphenyl]-2-propanesulfonamide,

2-[N-[2-(9H-carbazol-2-yloxy)ethyl]amino]-1-(3-nitrophenyl)ethanol,

N′-[3-[2-[2-(9H-carbazol-2-yloxy)ethylamino]1-hydroxyethyl]phenyl]-N,N-dimethylsulfamide,

2-[N-[2-(9H-carbazol-2-yloxy)ethyl]amino]-1-(3-aminophenyl)ethanol,

2-[N-[2-(9H-carbazol-2-yloxy)ethyl]amino]-1-[3-(hydroxymethyl)-4-hydroxyphenyl]ethanol,

N-[5-[2-[2-(9H-carbazol-2-yloxy)ethylamino]-1-hydroxyethyl]-3-hydroxyphenyl]methanesulfonamide,

N-[3-[2-[2-(9H-carbazol-2-yloxy)ethylamino]-1-hydroxyethyl]-2-hydroxyphenyl]methanesulfonamide,

N-[3-[2-[2-(9H-carbazol-2-yloxy)ethylamino]-1-hydroxyethyl]-4-hydroxyphenyl]methanesulfonamide,

(R)-N′-[5-[2-[2-(9H-carbazol-2-yloxy)ethylamino]-1-hydroxyethyl]-2-hydroxyphenyl]-N,N-dimethylsulfamide,

(S)-N′-[5-[2-[2-(9H-carbazol-2-yloxy)ethylamino]-1-hydroxyethyl]-2-hydroxyphenyl]-N,N-dimethylsulfamide,

N-[3-[2-[2-(6-acetylamino-9H-carbazol-2-yloxy)ethylamino]-1-hydroxyethyl]phenyl]methanesulfonamide,

N-[5-[2-[2-(6-acetylamino-9H-carbazol-2-yloxy)ethylamino]-1-hydroxyethyl]-2-hydroxyphenyl]methanesulfonamide,

(R)-N-[5-[2-[2-(9H-carbazol-2-yloxy)ethylamino]-1-hydroxyethyl]-2-fluorophenyl]methanesulfonamide,

(S)-N-[5-[2-[2-(9H-carbazol-2-yloxy)ethylamino]-1-hydroxyethyl]-2-fluorophenyl]methanesulfonamide,

(R)-N-[5-[2-[2-(9H-carbazol-2-yloxy)ethylamino]-1-hydroxyethyl]-2-chlorophenyl]methanesulfonamide,

(S)-N-[5-[2-[2-(9H-carbazol-2-yloxy)ethylamino]-1-hydroxyethyl]-2-chlorophenyl]methanesulfonamide,

N,N-dimethyl-[5-[2-[2-(9H-carbazol-2-yloxy)ethylamino]-1-hydroxyethyl]-2-hydroxy]benzenesulfonamide,

N-[5-[2-[2-(9H-carbazol-2-yloxy)ethylamino]-1-hydroxyethyl]-2-iodophenyl]methanesulfonamide,

N′-[5-[2-[2-(9H-carbazol-2-yloxy)ethylamino]-1-hydroxyethyl]-2-fluorophenyl]-N,N-dimethylsulfamide,

N′-[5-[2-[2-(9H-carbazol-2-yloxy)ethylamino]-1-hydroxyethyl]-2-chlorophenyl]-N,N-dimethylsulfamide,

(R)-N-methyl-[5-[2-[2-(9H-carbazol-2-yloxy)ethylamino]-1-hydroxyethyl]-2-hydroxy]benzenesulfonamide,

(R)-N-[5-[2-[2-(9H-carbazol-2-yloxy)ethylamino]-1-hydroxyethyl]-2-(hydroxymethyl)phenyl]methanesulfonamide

(R)-N-[3-[2-[2-(dibenzofuran-3-yloxy)ethylamino]-1-hydroxyethyl]phenyl]methanesulfonamide,

N′-[5-[2-[2-(dibenzofuran-3-yloxy)ethylamino]-1-hydroxyethyl]-2-hydroxyphenyl]-N,N-dimethylsulfamide,

(R)-N′-[5-[2-[2-(dibenzofuran-3-yloxy)ethylamino]-1-hydroxyethyl]-2-hydroxyphenyl]-N,N-dimethylsulfamide,

(S)-N′-[5-[2-[2-(dibenzofuran-3-yloxy)ethylamino]-1-hydroxyethyl]-2-hydroxyphenyl]-N,N-dimethylsulfamide,

N-[5-[2-[2-(dibenzofuran-3-yloxy)ethylamino]-1-hydroxyethyl]-2-fluorophenyl]methanesulfonamide,

N-[5-[2-[2-(dibenzofuran-3-yloxy)ethylamino]-1-hydroxyethyl]-2-chlorophenyl]methanesulfonamide,

N-[5-[2-[2-(dibenzothiophen-3-yloxy)ethylamino]-1-hydroxyethyl]-2-hydroxyphenyl]methanesulfonamide,

N′-[5-[2-[2-(dibenzothiophen-3-yloxy)ethylamino]-1-hydroxyethyl]-2-hydroxyphenyl]-N,N-dimethylsulfamide,

N-[3-[2-[2-(dibenzothiophen-3-yloxy)ethylamino]-1-hydroxyethyl]phenyl]methanesulfonamide,

(R)-N-[5-[2-[2-(dibenzothiophen-3-yloxy)ethylamino]-1-hydroxyethyl]-2-hydroxyphenyl]methanesulfonamide,

N-[5-[2-[2-(dibezothiophen-3-yloxy)ethylamino]-1-hydroxyethyl]-2-fluorophenyl]methanesulfonamide,

N-[5-[2-[2-(dibenzothiophen-3-yloxy)ethylamino]-1-hydroxyethyl]-2-chlorophenyl]methanesulfonamide,

N-[5-[2-[2-(7-aminofluoren-2-yloxy)ethylamino]-1-hydroxyethyl]-2-hydroxyphenyl]methanesulfonamide,

N′[5-[2-[2-(7-acetylaminofluoren-2-yloxy)ethylamino]-1-hydroxyethyl]-2-hydroxyphenyl]-N,N-dimethylsulfamide,

N′-[5-[2-[2-(9H-carbazol-2-yloxy)ethylamino]-1-hydroxyethyl]-2-aminophenyl]-N-benzyl-N-methylsulfamide,

N-[5-[2-[2-(9H-carbazol-2-yloxy)ethylamino]-1-hydroxyethyl]-2-aminophenyl]methanesulfonamide,

N-[5-[2-[2-(9H-carbazol-2-yloxy)ethylamino]-1-hydroxyethyl]-2-hydroxymethylphenyl]methanesulfonamide,

N-[5-[2-[2-(9H-carbazol-2-yloxy)ethylamino]-1-hydroxyethyl]-2-bromophenyl]methanesulfonamide,

N′-[5-[2-[2-(9H-carbazol-2-yloxy)ethylamino]-1-hydroxyethyl]-2-hydroxyphenyl]-N-benzyl-N-methylsulfamide,

N′-[5-[2-[2-(9H-carbazol-2-yloxy)ethylamino]-1-hydroxyethyl]-2-hydroxyphenyl]-N,N-diethylsulfamide,

N-[5-[2-[2-(9H-carbazol-2-yloxy)ethylamino]-1-methoxyethyl]-2-aminophenyl]methanesulfonamide,

N-[5-[2-[2-(9H-carbazol-2-yloxy)ethylamino]-1-methoxyethyl]-2-hydroxyphenyl]methanesulfonamide and

N-[5-[2-[2-(dibenzothiophen-3-yloxy)ethylamino]-1-methoxyethyl]-2-hydroxyphenyl]methanesulfonamide.

2-[N-[2-(9H-carbazol-2-yloxy)ethyl]amino]-1-(4-hydroxyphenyl)ethanol,

2-[N-[2-(9H-carbazol-2-yloxy)ethyl]amino]-1-(2-fluorophenyl)ethanol,

2-[N-[2-(9H-carbazol-2-yloxy)ethyl]amino]-1-(2-hydroxyphenyl)ethanol,

(R,R)-2-[N-[1-(9H-carbazol-2-yloxy)propan-2-yl]amino]-1-phenyl]ethanol,

[2-[N-[2-(9H-carbazol-2-yloxy)ethyl]amino]-1-phenyl]ethanol,

(R)-[2-[N-[2-(9H-carbazol-2-yloxy)ethyl]amino]-1-phenyl]ethanol,

(S)-[2-[N-[2-(9H-carbazol-2-yloxy)ethyl]amino]-1-phenyl]ethanol,

[2-[N-[2-(3-acetylamino-9H-carbazol-2-yloxy)ethyl]amino]-1-phenyl]ethanol,

[2-[N-[2-(3-amino-9H-carbazol-2-yloxy)ethyl]amino]-1-phenyl]ethanol,

[2-[N-[2-(3-hydroxy-9H-carbazol-2-yloxy)ethyl]amino]-1-phenyl]ethanol

[2-[N-[2-(6-amino-9H-carbazol-2-yloxy)ethyl]amino]-1-phenyl]ethanol,

[2-[N-[2-(6-acetylamino-9H-carbazol-2-yloxy)ethyl]amino]-1-phenyl]ethanol,

[2-[N-[1-(9H-carbazol-2-yloxy)propan-2-yl]amino]-1-phenyl]ethanol and

[2-[N-[2-(dibenzofuran-3-yloxy)ethyl]amino]-1-phenyl]ethanol.

N-[5-[2-[2-(9H-carbazol-2-yloxy)ethylamino]-1-methoxyethyl]-2-hydroxyphenyl]methanesulfonamide,

N-[5-[2-[2-(dibenzothiophen-3-yloxy)ethylamino]-1-methoxyethyl]-2-hydroxyphenyl]methanesulfonamide,

N-[5-[2-[2-(9H-carbazol-2-yloxy)ethylamino]-1-methoxyethyl]-2-aminophenyl]methanesulfonamide and

N-[5-[2-[2-(9H-carbazol-2-yloxy)ethylamino]-1-methoxyethyl]-2-chlorophenyl]methanesulfonamide; or a salt thereof

Other suitable beta agonists include the compounds of U.S. Pat. No. 5,786,356 and European published patent application number EP 764640A, especially the specific examples therein including LY-377604.

Suitable beta agonists also include the compounds of WO 9616938 and EP 801059, especially the specific examples therein including 2-[3-(7-carboxymethoxyindol-3-yl)-(2R)-2-propylamino]-(1R)-1-(3-chlorophenyl)ethanol (AD-9677) and derivatives thereof. The disclosures of each of these documents is incorporated herein by reference, with particular reference to the methods of preparation and pharmaceutically acceptable derivatives of the compounds therein.

With reference to EP 801059, further particular beta agonists included herein are those selected from the list consisting of:

2-[3-(7-methoxyindol-3-yl)-2-propylamino]-1-(3-chlorophenyl)ethanol;

2-[3-(7-ethoxyindol-3-yl)-2-propylamino]1-(3-chlorophenyl)ethanol;

2-[3-(7-methoxycarbonylmethoxyindol-3-yl)-2-propylamino]-1-(3-chlorophenyl)ethanol;

2-[3-(7-carboxymethoxyindol-3-yl)-2-propylamino]-1-(3-chlorophenyl)ethanol; and derivatives thereof.

It will be understood that the beta agonist and the other antidabetic agent are each administered in a pharmaceutically acceptable form, including pharmaceutically acceptable derivatives such as pharmaceutically acceptable salts, esters and solvates thereof, as appropriate of the relevant pharmaceutically active agent. In certain instances herein the names used for the other antidabetic agent may relate to a particular pharmaceutical form of the relevant active agent: It will be understood that all pharmaceutically acceptable forms of the active agents per se are encompassed by this invention.

Suitable pharmaceutically acceptable forms of the other antidiabetic agent depend upon the particular agent being used but include known pharmaceutically acceptable forms of the particular agent chosen. Such derivatives are found or are referred to in standard reference texts such as the British and US Pharmacopoeias, Remington's Pharmaceutical Sciences (Mack Publishing Co.), Martindale The Extra Pharmacopoeia (London, The Pharmaceutical Press) (for example see the 31st Edition page 341 and pages cited therein) or the above mentioned publications.

Suitable pharmaceutically acceptable forms of the beta agonist of formula (I), including salted forms and solvated forms, include those described in WO97/25311 and EP0882707A1.

The beta agonist is prepared according to published methods, for example when the beta agonist is a compound of formula (1) of WO97/25311 and EP0882707A1, or a derivative thereof such as a pharmaceutically acceptable salt thereof or a pharmaceutically acceptable solvate thereof, then it is prepared according to methods disclosed therein.

Certain of the compounds mentioned herein, for example the beta agonists of WO97/25311, EP0882707A1, U.S. Pat No. 5,786,356, EP 764640A, WO 9616938 and EP 801059 and the thiazolidinediones, may contain one or more chiral carbon atoms and hence can exist in two or more isomeric forms, all of which are encompassed by the invention, either as individual isomers or as mixtures of isomers, including racemates. Certain of the compounds mentioned herein, in particular the thiazolidinediones such as Compound (I), may exist in one of several tautomeric forms, all of which are encompassed by the invention as individual tautomeric forms or as mixtures thereof

The beta agonist and the other antidiabetic agent of choice is prepared according to known methods, such methods are found or are referred to in standard reference texts, such as the British and US Pharmacopoeias, Remington's Pharmaceutical Sciences (Mack Publishing Co.), Martindale The Extra Pharmacopoeia (London, The Pharmaceutical Press) (for example see the 31st Edition page 341 and pages cited therein) or the above mentioned publications.

When used herein the term ‘conditions associated with diabetes’ includes those conditions associated with the pre-diabetic state, conditions associated with diabetes mellitus itself and complications associated with diabetes mellitus.

When used herein the term ‘conditions associated with the pre-diabetic state’ includes conditions such as insulin resistance, including hereditary insulin resistance, impaired glucose tolerance and hyperinsulinaemia.

‘Conditions associated with diabetes mellitus itself’ include hyperglycaemia, insulin resistance, including acquired insulin resistance and obesity. Further conditions associated with diabetes mellitus itself include hypertension and cardiovascular disease, especially atherosclerosis and conditions associated with insulin resistance. Conditions associated with insulin resistance include polycystic ovarian syndrome and steroid induced insulin resistance and gestational diabetes.

‘Complications associated with diabetes mellitus’ includes renal disease, especially renal disease associated with Type 2 diabetes, neuropathy and retinopathy.

Renal diseases associated with Type 2 diabetes include nephropathy, glomerulonephritis, glomerular sclerosis, nephrotic syndrome, hypertensive nephrosclerosis and end stage renal disease.

As used herein the term ‘pharmaceutically acceptable’ embraces both human and veterinary use: for example the term ‘pharmaceutically acceptable’ embraces a veterinarily acceptable compound.

Diabetes mellitus is preferably Type 2 diabetes.

Suitably, the particularly beneficial effect on glycaemic control provided by the treatment of the invention is an improved therapeutic ratio for the combination of the invention relative to the therapeutic ratio for one compound of the combination when used alone and at a dose providing an equivalent efficacy to the combination of the invention.

In a preferred aspect, the particularly beneficial effect on glycaemic control provided by the treatment of the invention is indicated to be a synergistic effect relative to the control expected from the effects of the individual active agents.

In a further aspect of the invention, combining doses of the beta agonist and the other agent will produce a greater beneficial effect than can be achieved for either agent alone at a dose twice that used for that agent in the combination.

Glycaemic control may be characterised using conventional methods, for example by measurement of a typically used index of glycaemic control such as fasting plasma glucose or glycosylated haemoglobin (Hb A1c). Such indices are determined using standard methodology, for example those described in: Tuescher A, Richterich, P., Schweiz. med. Wschr. 101 (1971), 345 and 390 and Frank P., ‘Monitoring the Diabetic Patent with Glycosolated Hemoglobin Measurements’, Clinical Products 1988.

In a preferred aspect, the dosage level of each of the active agents when used in accordance with the treatment of the invention will be less than would have been required from a purely additive effect upon glycaemic control.

It is also considered that the treatment of the invention will effect an improvement, relative to the individual agents, in the levels of advanced glycosylation end products (AGEs), and serum lipids including total cholesterol, HDL-cholesterol, LDL-cholesterol including improvements in the ratios thereof, in particular an improvement in serum lipids including total cholesterol, HDL-cholesterol, LDL-cholesterol including improvements in the ratios thereof.

As indicated above it is also considered that treatment with the combination of the beta agonist and the other antidiabetic agents (especially sulphonylureas, insulin sensitisers or insulin) will significantly reduce, preferably remove, the body weight increasing effects of the other antidiabetic agents.

It is also considered that the thermogenic effects of the beta-3 agonist will be enhanced in the combination treatment of the invention. Thus, for example the weight reducing effects of the beta-3 agonist will be enhanced in the combination treatment of the invention.

In the treatment of the invention, the active medicaments are preferably administered in pharmaceutical composition form. As indicated above, such compositions can include both medicaments or one only of the medicaments.

Accordingly, in one aspect the present invention also provides a pharmaceutical composition comprising a beta agonist and another antidiabetic agent and a pharmaceutically acceptable carrier therefor.

Thus, in a further aspect, the invention also provides a process for preparing a pharmaceutical composition comprising a beta agonist, another antidiabetic agent and a pharmaceutically acceptable carrier therefor, which process comprises admixing the beta agonist, another antidiabetic agent and a pharmaceutically acceptable carrier.

The compositions are preferably in a unit dosage form in an amount appropriate for the relevant daily dosage.

Suitable dosages, including especially unit dosages, of the beta agonist or the other antidiabetic agent include the known dosages including unit doses for these compounds as described or referred to in reference text such as the British and US Pharmacopoeias, Remington's Pharmaceutical Sciences (Mack Publishing Co.), Martindale The Extra Pharmacopoeia (London, The Pharmaceutical Press) (for example see the 31st Edition page 341 and pages cited therein) or the above mentioned publications.

Thus, suitable dosages for the beta agonists of WO97/25311 and EP0882707A1 include those disclosed therein, for example 0.01 to 2000 mg per day. Also, the suitable doses of the other beta agonists mentioned herein include those mentioned in the relevant publications mentioned above, for example suitable AD-9677 doses include those disclosed in WO 9616938 and EP 801059, such as in the range of 0.01-20 mg/kg/day including 0.05-10 mg/kg/day.

For the alpha glucosidase inhibitor, a suitable amount of acarbose is in the range of from 25 to 600 mg, including 50 to 600 mg, for example 100 mg or 200 mg.

For the biguanide, a suitable dosage of metformin is between 100 to 3000 mg, for example 250, 500 mg, 850 mg or 1000 mg.

For the insulin secretagogue, a suitable amount of glibenclamide is in the range of from 2.5 to 20 mg, for example 10 mg or 20 mg; a suitable amount of glipizide is in the range of from 2.5 to 40 mg; a suitable amount of gliclazide is in the range of from 40 to 320 mg; a suitable amount of tolazamide is in the range of from 100 to 1000 mg; a suitable amount of tolbutamide is in the range of from 1000 to 3000 mg; a suitable amount of chlorpropamide is in the range of from 100 to 500 mg; and a suitable amount of gliquidone is in the range of from 15 to 180 mg. Also a suitable amount of glimepiride is 1 to 6mg and a suitable amount of glipentide is 2.5 to 20 mg.

A suitable amount of repaglinide is in the range of from 0.5 mg to 20 mg, for example 16 mg. Also a suitable amount of nateglinide is 90 to 360 mg, for example 270 mg.

In one particular aspect, the composition comprises 2 to 12 mg of Compound (1).

Suitably the composition comprises 2, 3, 4, 5, 6, 7, 8, 9, 10, 11 or 12 mg of Compound (I).

Particularly, the composition comprises 2 to 4, 4 to 8 or 8 to 12 mg of Compound (I).

Particularly, the composition comprises 2 to 4 mg of Compound (I).

Particularly, the composition comprises 4 to 8 mg of Compound (I).

Particularly, the composition comprises 2 to 1 mg of Compound (I).

Particularly, the composition comprises 8 to 12 mg of Compound (I).

Preferably, the composition comprises 2 mg of Compound (I).

Preferably, the composition comprises 4 mg of Compound (I).

Preferably, the composition comprises 8 mg of Compound (I).

Suitable unit dosages of other insulin sensitisers include from 100 to 800 mg of troglitazone such as 200, 400, 600 or 800 mg or from 5 to 50 mg, including 10 to 40 mg, of pioglitazone, such as 20, 30 or 40 mg and also including 15, 30 and 45 mg of pioglitazone.

In the treatment the medicaments may be administered from 1 to 6 times a day, but most preferably 1 or 2 times per day.

Also, the dosages of each particular active agent in any given composition can as required vary within a range of doses known to be required in respect of accepted dosage regimens for that compound. Dosages of each active agent can also be adapted as required to take into account advantageous effects of combining the agents as mentioned herein.

It will be understood that the beta agonist and the other antidiabetic agent are in a pharmaceutically acceptable form, including pharmaceutically acceptable derivatives such as pharmaceutically acceptable salts, esters and solvates thereof, as appropriate to the relevant pharmaceutically active agent chosen. In certain instances herein the names used for the antidiabetic agent may relate to a particular pharmaceutical form of the relevant active agent: It will be understood that all pharmaceutically acceptable forms of the active agents per se are encompassed by this invention.

The present invention also provides a pharmaceutical composition comprising a beta agonist, another antidiabetic agent and a pharmaceutically acceptable carrier therefor, for use as an active therapeutic substance.

In particular, the present invention provides a pharmaceutical composition comprising a beta agonist, another antidiabetic agent and a pharmaceutically acceptable carrier therefor, for use in the treatment of diabetes mellitus, especially Type 2 diabetes and conditions associated with diabetes mellitus.

Usually the compositions are adapted for oral administration. However, they may be adapted for other modes of administration, for example parenteral administration, sublingual or transdermal administration.

The compositions may be in the form of tablets, capsules, powders, granules, lozenges, suppositories, reconstitutable powders, or liquid preparations, such as oral or sterile parenteral solutions or suspensions.

In order to obtain consistency of administration it is preferred that a composition of the invention is in the form of a unit dose.

Unit dosage presentation forms for oral administration may be in tablet or capsule form and may as necessary contain conventional excipients such as binding agents, fillers, lubricants, glidants, disintegrants and wetting agents.

The solid oral compositions may be prepared by conventional methods of blending, filling or tabletting. Repeated blending operations may be used to distribute the active agent throughout those compositions employing large quantities of fillers. Such operations are of course conventional in the art. The tablets may be coated according to methods well known in normal pharmaceutical practice, in particular with an enteric coating.

Oral liquid preparations may be in the form of, for example, emulsions, syrups, or elixirs, or may be presented as a dry product for reconstitution with water or other suitable vehicle before use. Such liquid preparations may contain conventional additives such as suspending agents, for example sorbitol, syrup, methyl cellulose, gelatin, hydroxyethylcellulose, carboxymethylcellulose, aluminium stearate gel, hydrogenated edible fats; emulsifying agents, for example lecithin, sorbitan monooleate, or acacia; non-aqueous vehicles (which may include edible oils), for example almond oil, fractionated coconut oil, oily esters such as esters of glycerine, propylene glycol, or ethyl alcohol; preservatives, for example methyl or propyl p-hydroxybenzoate or sorbic acid; and if desired conventional flavouring or colouring agents.

For parenteral administration, fluid unit dosage forms are prepared utilizing the compound and a sterile vehicle, and, depending on the concentration used, can be either suspended or dissolved in the vehicle. In preparing solutions the compound can be dissolved in water for injection and filter sterilized before filling into a suitable vial or ampoule and sealing. Advantageously, adjuvants such as a local anaesthetic, a preservative and buffering agent can be dissolved in the vehicle. To enhance the stability, the composition can be frozen after filling into the vial and the water removed under vacuum. Parenteral suspensions are prepared in substantially the same manner, except that the active compound is suspended in the vehicle instead of being dissolved, and sterilization cannot be accomplished by filtration. The compound can be sterilized by exposure to ethylene oxide before suspending in the sterile vehicle. Advantageously, a surfactant or wetting agent is included in the composition to facilitate uniform distribution of the compound.

Compositions may contain from 0.1% to 99% by weight, preferably from 10-60% by weight, of the active material, depending upon the method of administration.

Examples of binding agents include acacia, alginic acid, carboxymethylcellulose calcium, carboxymethylcellulose sodium, dextrates, dextrin, dextrose, ethylcellulose, gelatin, liquid glucose, guar gum, hydroxyethyl cellulose, hydroxypropyl cellulose, hydroxypropyl methylcellulose, magnesium aluminium silicate, maltodextrin, methyl cellulose, polymethacrylates, polyvinylpyrrolidone, pregelatinised starch, sodium alginate, sorbitol, starch, syrup, tragacanth.

Examples of fillers include calcium carbonate, calcium phosphate, calcium sulphate, carboxymethylcellulose calcium, carboxymethylcellulose sodium, compressible sugar, confectioner's sugar, dextrates, dextrin, dextrose, dibasic calcium phosphate dihydrate, dibasic calcium phosphate, fructose, glyceryl palmitostearate, glycine, hydrogenated vegetable oil-type 1, kaolin, lactose, maize starch, magnesium carbonate, magnesium oxide, maltodextrin, mannitol, microcrystalline cellulose, polymethacrylates, potassium chloride, powdered cellulose, pregelatinised starch, sodium chloride, sorbitol, starch, sucrose, sugar spheres, talc, tribasic calcium phosphate, xylitol.

Examples of lubricants include calcium stearate, glyceryl monostearate, glyceryl palmitostearate, magnesium stearate, microcrystalline cellulose, sodium benzoate, sodium chloride, sodium lauryl sulphate, stearic acid, sodium stearyl fumarate, talc, zinc stearate.

Examples of glidants include colloidal silicon dioxide, powdered cellulose, magnesium trisilicate, silicon dioxide, talc.

Examples of disintegrants include alginic acid, carboxymethylcellulose calcium, carboxymethylcellulose sodium, colloidal silicon dioxide, croscarmellose sodium, crospovidone, guar gum, magnesium aluminium silicate, microcrystalline cellulose, methyl cellulose, polyvinylpyrrolidone, polacrilin potassium, pregelatinised starch, sodium alginate, sodium lauryl sulphate, sodium starch glycollate.

An example of a pharmaceutically acceptable wetting agent is sodium lauryl sulphate.

The compositions are prepared and formulated according to conventional methods, such as those disclosed in standard reference texts, for example the British and US Pharmacopoeias, Remington's Pharmaceutical Sciences (Mack Publishing Co.), Martindale The Extra Pharmacopoeia (London, The Pharmaceutical Press) (for example see the 31st Edition page 341 and pages cited therein) and Harry's Cosmeticology (Leonard Hill Books) or the above mentioned publications.

For example, the solid oral compositions may be prepared by conventional methods of blending, filling or tabletting. Repeated blending operations may be used to distribute the active agent throughout those compositions employing large quantities of fillers. Such operations are of course conventional in the art. The tablets may be coated according to methods well known in normal pharmaceutical practice.

Compositions may, if desired, be in the form of a pack accompanied by written or printed instructions for use.

No adverse toxicological effects are expected for the compositions or methods of the invention in the above mentioned dosage ranges.

1. A method for the treatment of diabetes mellitus and conditions associated with diabetes mellitus in a mammal such as a human, which method comprises administering an effective, non-toxic and pharmaceutically acceptable amount of a beta agonist and another antidiabetic agent, to a mammal in need thereof.

2. A method according to claim 1, wherein the other antidiabetic agent is selected from an alpha glucosidase inhibitor, a biguanide, an insulin secretagogue, an insulin sensitiser and insulin.

3. A method according to claim 1 or claim 2, wherein the other antidiabetic agent is an alpha glucosidase inhibitor.

4. A method according to claim 3, wherein the alpha glucosidase inhibitor is selected from acarbose, emiglitate, miglitol and voglibose.

5. A method according to claim 1 or claim 2, wherein the other antidiabetic agent is a biguanide.

6. A method according to claim 5, wherein the biguanide is selected from metformin, buformin and phenformin.

7. A method according to claim 1 or claim 2, wherein the other antidiabetic agent is an insulin secretagogue.

8. A method according to claim 7, wherein the insulin secretagogue is a sulphonylurea.

9. A method according to claim 7 or claim 8, wherein the sulphonylurea is selected from glibenclamide, glipizide, gliclazide, glimepiride, tolazamide, tolbutamide, acetohexamide, carbutamide, chlorpropamide, glibomuride, gliquidone, glisentide, glisolamide, glisoxepide, glyclopyamide, glycylamide and glipentide.

10. A method according to claim 1 or claim 2, wherein the other antidiabetic agent is an insulin sensitiser

11. A method according to claim 10, wherein the insulin sensitiser is Compound (I) or a derivative thereof.

12. A method according to claim 10 or 11, wherein the insulin sensitiser is Compound (I) or a derivative thereof.

13. A method according to claim 10 or 11, wherein the insulin sensitiser is (+)-5-[[4-[(3,4-dihydro-6-hydroxy-2,5,7,8-tetramethyl-2H-1-benzopyran-2-yl)methoxy]phenyl]methyl]-2,4-thiazolidinedione (or troglitazone), 5-[4-[(1-methylcyclohexyl)methoxy]benzyl]thiazolidine-2,4-dione (or ciglitazone), 5-[4-[2-(5-ethylpyridin-2-yl)ethoxy]benzyl]thiazolidine-2,4-dione (or pioglitazone) or 5-[(2-benzyl-2,3-dihydrobenzopyran)-5-ylmethyl)thiazolidine-2,4-dione (or englitazone);) or a derivative thereof.

14. A method according to any one of claims 1 to 13, wherein the beta agonist is a compound of formula (I) of WO/9725311.

15. A method according to any one of claims 1 to 14, wherein the beta agonist is selected from the list consisting of examples of WO/9725311

16. A pharmaceutical composition, comprising a beta agonist and another antidiabetic agent and a pharmaceutically acceptable carrier therefor.

17. A composition according to claim 16, wherein the other antidiabetic agent is selected from an alpha glucosidase inhibitor, a biguanide, an insulin secretagogue or an insulin sensitiser.

18. A composition according to claim 17, in unit dosage form
